SEQUENCE LISTING (14 sequences)

SEQ ID NO: 1 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 2 - DNA, 1395 bp, Homo sapiens
atgattgttttcgtcagattcaatagttcccacgggttccctgtcgaggtggacagtgatactagcatcttccagctgaaagaagtggtggcgaagcggcagggagttcctgcagaccaactgagggtcattttcgccggcaaggagctgaggaacgattggactgtgcagaactgtgaccttgatcagcagagtatcgttcacatagtgcagcgcccgtggaggaaggggcaggagatgaacgcaacgggcggggacgaccccagaaatgctgcggggggttgcgagcgggaacctcagtctctgactcgggtggacctgtctagctctgtgctcccaggtgatagcgttggcctcgctgttatcctgcatacagactccaggaaggatagtcctcccgccgggtctcctgccggccgaagtatctataactcattttacgtttactgcaaaggaccctgccaacgcgtacaacccggcaagctccgcgtgcaatgctcaacttgtaggcaggccacactcactttgacgcaaggtccctcttgctgggacgatgtgctgattccgaatagaatgagtggcgagtgccaatcaccccattgtcccggtacaagcgcggaattcttcttcaaatgcggcgcacatcccacgtcagacaaagagacttcagtcgctctccacctgatagccaccaactcccgcaacattacctgtataacttgcacggatgtccgctcccccgtgttggtgttccagtgtaactccagacatgtgatctgtctggactgctttcacctgtactgcgtgactagacttaatgacagacagtttgtacatgacccccagctgggatacagcctgccgtgcgtggccggttgtcccaacagcctgattaaggagctgcaccatttcaggatcctgggcgaggagcagtacaacagataccagcagtacggggcggaggagtgtgttcttcagatggggggggtgctgtgccccaggcccggctgcggtgctggtctgttgccagagcccgaccagagaaaggtcacatgtgagggcggtaatgggcttggctgtggatttgccttctgcagggaatgtaaagaggcctaccacgagggcgaatgcagtgccgttttcgaagcaagtggcaccacaacacaggcctatagagttgatgaaagggcagcagaacaagcgaggtgggaagccgcctccaaagaaactatcaaaaaaacgacaaagccatgccccaggtgccatgtgcctgtggagaaaaacgggggatgcatgcatatgaaatgtccccagccccagtgccggttggagtggtgttggaactgtggctgcgaatggaatcgggtctgcatgggggaccactggtttgacgtg

SEQ ID NO: 3 - DNA, 1398 bp, Homo sapiens
atgattgtgttcgttcgattcaattcatcccatggatttccagtcgaggtcgattcagatacctccatattccagctcaaagaagttgtcgcaaagaggcaaggagtgccagccgaccagctgcgagtcatctttgccggaaaggagttgaggaacgactggaccgttcaaaattgtgacctggaccagcagtcaatagtgcacatcgtgcaaaggccttggcggaagggtcaagagatgaacgctactggtggcgacgatcctcggaatgcagcaggcggctgcgaacgagagcctcagagccttaccagggtagatttgtcatccagcgtattgcctggtgactcagtaggactggctgtaattcttcatacagacagcaggaaagatagcccaccagccggcagccccgctggtagaagtatctacaactcattctacgtctattgcaaagggccgtgtcagcgggtgcaaccgggtaaactcagagtccaatgcagcacctgtagacaagctacactgacacttacacaagggcctagttgttgggacgacgttcttattcccaatagaatgtcaggtgagtgtcaaagtcctcattgtccggggactagtgctgagttttttttcaaatgcggcgctcaccccactagtgacaaggagacaagcgtggccctgcatctcatagcgacgaatagcagaaacataacatgcatcacttgcacggacgttcggtcacctgtgcttgtgtttcaatgtaacagccggcatgtcatttgtcttgattgctttcacctctactgtgtgacacgcttgaatgacagacaattcgtccatgacccacaattgggatacagtttgccctgcgtagcgggttgtccaaattctttgattaaggagctgcatcactttcggatcctgggagaagagcagtacaatcgataccagcagtatggagctgaagagtgtgtgctccaaatgggcggggttctttgtccccggcctggctgcggcgccggtttgctccccgaaccagatcagcggaaagttacatgtgagggtggaaatggtcttggctgtggcttcgcgttctgccgggagtgcaaagaagcgtaccatgaaggggagtgcagcgcagtttttgaggcaagtggcacgacgacccaggcttaccgggtagacgaacgcgcagcagagcaggccagatgggaagcggcctccaaggagaccattaaaaagacaaccaaaccttgtcctcggtgtcacgtgcccgtcgagaagaacgggggctgtatgcatatgaaatgcccacaaccgcaatgtaggctggaatggtgttggaactgcggctgcgaatggaatagggtgtgtatgggagaccattggtttgacgtctag

SEQ ID NO: 4 - DNA, 1398 bp, Homo sapiens
atgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtag

SEQ ID NO: 5 - DNA, 145 bp, Homo sapiens
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 6 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETPVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 7 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 8 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 9 - protein, 449 aa, Homo sapiens
MELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 10 - protein, 487 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 11 - protein, 274 aa, Homo sapiens
MGKSLSHLPLHSSKEDAYDGVTSENMRNGLVNSEVHNEDGRNGDVSQFPYVEFTGRDSVTCPTCQGTGRIPRGQENQLVALIPYSDQRLRPRRTKLYVMASVFVCLLLSGLAVFFLFPRSIDVKYIGVKSAYVSYDVQKRTIYLNITNTLNITNNNYYSVEVENITAQVQFSKTVIGKARLNNITIIGPLDMKQIDYTVPTVIAEEMSYMYDFCTLISIKVHNIVLMMQVTVTTTYFGHSEQISQERYQYVDCGRNTTYQLGQSEYLNVLQPQQ

SEQ ID NO: 12 - protein, 517 aa, Homo sapiens
MAALGPSSQNVTEYVVRVPKNTTKKYNIMAFNAADKVNFATWNQARLERDLSNKKIYQEEEMPESGAGSEFNRKLREEARRKKYGIVLKEFRPEDQPWLLRVNGKSGRKFKGIKKGGVTENTSYYIFTQCPDGAFEAFPVHNWYNFTPLARHRTLTAEEAEEEWERRNKVLNHFSIMQQRRLKDQDQDEDEEEKEKRGRRKASELRIHDLEDDLEMSSDASDASGEEGGRVPKAKKKAPLAKGGRKKKKKKGSDDEAFEDSDDGDFEGQEVDYMSDGSSSSQEEPESKAKAPQQEEGPKGVDEQSDSSEESEEEKPPEEDKEEEEEKKAPTPQEKKRRKDSSEESDSSEESDIDSEASSAFFMAKKKTPPKRERKPSGGSSRGNSRPGTPSAEGGSTSSTLRAAASKLEQGKRVSEMPAAKRLRLDTGPQSLSGKSTPQPPSGKTTPNSGDVQVTEDAVRRYLTRKPMTTKDLLKKFQTKKTGLSSEQTVNVLAQILKRLNPERKMINDKMHFSLKE

SEQ ID NO: 13 - DNA, 3793 bp, Homo sapiens
tctcattagaagtgaggcggggccggccaaatcgaatggacaccgggtaattagcagggttacccagatactccagcacctctttcccgtcggccgtgtacctgccattcacgtccatgccattgatggccagcactgcatgacccactgcagaggtgaagctaacggtcagcgaaggtgcagcccggggattccgccgaggggacaagggacccgacacaaccccttttcccccaaccccgcacctacaaccagcccacttctacagcactggggccctcccacccccgcacccgccacgggcccgagcctagcccacctcggatgccgtcccgctggccgaaagcaaccaacacacgctcatcgtgtagcttgagcagcagatccagcggataactgaaagttttctcagcctcagcccgtggcgcgtagctgtccaactggtaaatcaagccgccagctttgttcaccacatacacactaaaaatcgccatcgctgccttgccgctcggaaactggtattcagcctctacccgacggcccctccccggaaccgcatcacagcacttgccgccggccccaccccagcctcctcctcctcctcctcctcctcccgcgccccccgtgcagccacctgctgcacttgcgcactgggagcgacacgctcgggcataagtagtgccgaaaagttagctgccgagacctggtggattgcttttcgtttatcagtgcaggaaaacagcgctatagtactgcgtcacaactagcgcagactccggcagtatttaggcggtgcggcttgggaactagaatccacttcctgtcttccgcctcaggctagagggcgagcgcttcgccgtgggacttcttctgcctggctccgcctcttgccccggaagtactcacagcggacggtggtttttgggcccgtttctgagcagcgcttcctttttgtccgacatcttgacgaggctgcggtgtctgctgctattctccgagcttcgcaatggtaagcttcaggggtgtgaagtcgccggcgttcttgggtttgaggactcagtggggagagccttcggcgggagcgctccttggcctgccggcctcggttgcagggcgggcgcggttattgcttggcccatgtgctctggtggtggagtttgcgggggctgagggcgcagtattaggggactttggcgctatttgaggacctggttgcattcccgctgccctcctacagccgcctaaggacgacaagaagaagaaggacgctggaaagtcggccaagaaagacaaagacccagtgaacaaatccgggggcaaggccaaaaagaaggtagaaataagacctctctgaaagagactaggggtaactctctcgtaatcctctagtaataggtaacttgtatagtaagtggtttttcaggtgtagatttctagagtcaaaatgtgagagtttatcttcccgtcaccactcgttctttttcccattaggatcatgaaaatgggtctgttgtgcgaagtgtctgccgctgtgcctgctgtgttatttttaactgatctagtggggctcggcccctgtttgaaggccaaaaacgtgtcggtgttttttttttgtttttgttttagtaatgtgtaatttatccttgataacggtggaacagatttctctgacgcagattactcgagagggaaagggtgcttctgccagaaatactaacttgtttctgttttgttttggtgagcagaagtggtccaaaggcaaagttcgggacaagctcaataacttagtcttgtttgacaaagctacctatgataaactctgtaaggaagttcccaactataaacttataaccccagctgtggtctctgagagactgaagattcgaggctccctggccagggcagcccttcaggagctccttagtaaaggtgaggggtgtatcctacatgtgtgtttttgtaggttaaattgtcttgaccatgttaagcatcttcagtggttttgctggaaaagcagaattaaaaaaaaaaagcgtggcttgaccattggctgttagtaatgtaattctgacgtcttactcctgatcctgagatgaattctcagggttcttagccacttttgtgccgtggaccctgtggcagtttagtgaagcccaaggatcttttatgtttcgagtaaatggatgcatagaattacagggacaaccgtttttgaaataattagattactattttgaaacaactttgaaaatgtttaaaacctttatggtaaatattttgttgatgtattaaattttaaaaccagaaatttagtacggtctactcagtagtatggtctgattaccataattccacaataataaggctcagctaactatagtgactgaacgtctataattctagcactttgggaggccaaggcgggtgaatcaacggaggtcaggagttaaagaccagcctggccaatatggtgaaaacctgctctactgaaagttagctggacgtgggggcacacgtctgtaatcccagctactcaggatgctgaggcatgaggatcccttgaacccaggagatggaggtggcagtgagccgagatgacaccactgcactccagccttagtgacagcaaaagactgtctcagaaagggggggggggtggaagataatggagccctaatttaaaggaaaagtaaggatagatgatccgttaaaaacttggattctcggttaccgaacgtcagattaagcaattctggagccaggtgcagtggtacccttgtatttctagctacttgggaggccaaagcaggaggatcatttgagccaaggagttttaagaccattctgggcacctctgagagaactctgtctttttgttttccttttctttaaatagagatgcggttttgccatgttgcccaggctggtctcctgggctcaagagatccacctgtccaaagtgctgggattacaggcatgagcctctgcacccggccaaaacaaaccttactagagtctcattctgttgcccaggttggagtgcggaggggcagtcttggctcaatgcaaccaccaattcctgggttcaggtggtcctcacctcagcttcccaagtagctggaattacaagcatgtgccaccatgcccagctaatttttgtatttttggtagagatggggtttcaccttgttggccaggctggtgtgcaactccttacctcaagctatctgcccgtctccacctcccaaagcagtgggattataagcatgagccaccgcgcccagccaaaaaccttactagtttctattgtagcatctgttaagcatctcatcgtgctattctctccccctaggacttatcaaactggtttcaaagcacagagctcaagtaatttacaccagaaataccaagggtggagatgctccagctgctggtgaagatgcatgaataggtgagtaggaatgtgtgggctcatggtgtaggaggtagatacaaagctttatggttctgattcttttaatttttttttacaggtccaaccagctgtacatttggaaaaataaaactttattaaatcaaatgaatgagtatgtctgtttcctaagaaagacaatgataaagaatttggtggaaggtataataggggtttgttgactttgcttttagcctcatggtagttggtagagagcatgattagcttttttctgtatgtgactgcttcttcattgctgcagcttcagttttgaattgatgtctgaaaggaaataaagggttaacacgatgatgaagggtg

SEQ ID NO: 14 - DNA, 6762 bp, Homo sapiens
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

(19) United States
(12) Patent Application Publication
(10) Pub. No.: US 2023/0304036 A1
(43) Pub. Date: Sep. 28, 2023

(54) AAV VECTORS ENCODING PARKIN AND USES THEREOF

(71) Applicant: Prevail Therapeutics, Inc., New York, NY (US)

(72) Inventors: Asa Abeliovich, New York, NY (US); Benjamin Shykind, New York, NY (US)

(73) Assignee: Prevail Therapeutics, Inc., New York, NY (US)

(21) Appl. No.: 18/019,357

(22) PCT Filed: Aug. 3, 2021
(86) PCT No.: PCT/US2021/044351

§ 371 Date: Feb. 2, 2023

Related U.S. Application Data
(60) Provisional application No. 63/060,353, filed on Aug. 3, 2020.

(51) Int. Cl. C12N 15/86; A61K 48/00

(52) U.S. Cl. CPC C12N 15/86; A61K 48/0025; A61K 48/0066; C12N 2750/14143; C12N 2830/48

(57) ABSTRACT

The disclosure relates, in some aspects, to compositions and methods for delivery of transgenes to a subject. In some embodiments, the disclosure provides expression constructs (e.g., vectors containing an expression construct) comprising a transgene encoding human Parkin or a portion thereof. In some embodiments, the disclosure provides methods of treating a neurodegenerative disease (e.g., Parkinson’s disease) by administering such expression constructs to a subject in need thereof.

### RELATED APPLICATION

This application is a national stage filing under 35 U.S.C. § 371 of international PCT application PCT/US2021/044351, filed Aug. 3, 2021, which claims priority under 35 U.S.C. § 119(e) to U.S. provisional pat. application, U.S.S.N. 63/060,353, filed Aug. 3, 2020, the entire contents of each of which are incorporated by reference herein.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Jan. 25, 2023, is named P109470015US01-SEQ-KZM and is 49,372 bytes in size.

### BACKGROUND

Parkin (PRKN) is an E3 ubiquitin ligase that mediates clearance of damaged mitochondria from cells and also plays a role in cell survival by suppressing apoptosis. Mutations in PRKN have been observed to cause mitochondrial dysfunction and lead to neuronal death, Parkinson’s disease (PD), and tumorigenesis.

### SUMMARY

Aspects of the disclosure relate to compositions and methods for delivering a transgene to a subject. The disclosure is based, in part, on expression constructs (e.g., vectors) configured to express human Parkin (PRKN) protein encoded by a codon-optimized nucleic acid sequence. In some embodiments, expression constructs described herein reduce one or more signs or symptoms of a CNS disease (e.g., Parkinson’s disease) when administered to a subject.

Accordingly, in some aspects, the disclosure is based on an isolated nucleic acid comprising an expression construct encoding a human Parkin protein, wherein the human Parkin protein is encoded by a codon-optimized nucleic acid sequence.

In some embodiments, the human Parkin protein comprises the amino acid sequence set forth in SEQ ID NO: 1 or a portion thereof. In some embodiments, the codon-optimized nucleic acid sequence encoding the human protein comprises the sequence set forth in SEQ ID NO: 2 or 3. In some embodiments, a codon-optimized nucleic acid sequence does not comprise the nucleic acid sequence set forth in SEQ ID NO: 4.

In some embodiments, the expression construct further comprises a promoter operably linked to the codon-optimized nucleic acid sequence. In some embodiments, the promoter is a constitutive promoter, inducible promoter, or tissue-specific promoter. In some embodiments, the promoter is a chicken beta-actin (CBA) promoter, a CAG promoter, or a JeT promoter.

In some embodiments, the expression construct is flanked by adeno-associated virus (AAV) inverted terminal repeats (ITRs). In some embodiments, the AAV ITRs are of a serotype selected from the group consisting of AAV1 ITR, AAV2 ITR, AAV3 ITR, AAV4 ITR, AAV5 ITR, and AAV6 ITR. In some embodiments, the AAV ITRs are AAV2 ITR.

In some aspects, the disclosure provides a vector comprising an isolated nucleic acid as described herein. In some embodiments, the vector is a plasmid. In some embodiments, the vector is a viral vector. In some embodiments, the viral vector is a recombinant AAV (rAAV) vector or a Baculovirus vector.

In some aspects, the disclosure provides a host cell comprising an isolated nucleic acid or vector as described herein. In some embodiments, the host cell is a mammalian cell, yeast cell, bacterial cell, or insect cell. In some embodiments, the host cell is a human cell.

In some aspects, the disclosure provides a recombinant adeno-associated virus (rAAV) comprising: (i) a capsid protein; and (ii) an isolated nucleic acid or the vector as described herein. In some embodiments, the capsid protein is capable of crossing the blood-brain barrier. In some embodiments, the capsid protein is an AAV9 capsid protein or a variant thereof. In some embodiments, the rAAV transduces neuronal cells and/or non-neuronal cells of the central nervous system (CNS).

In some aspects, the disclosure provides a composition comprising an isolated nucleic acid, vector, host cell, or rAAV as described herein. In some embodiments, the composition is a pharmaceutical composition. In some embodiments, the pharmaceutical composition further comprises a pharmaceutically acceptable carrier.

In some aspects, the disclosure provides a method for delivering a transgene to cells of the central nervous system, the method comprising administering an expression construct (e.g., rAAV) as described herein to a subject. In some embodiments, the administration is direct injection into CNS tissue. In some embodiments, the administration is peripheral administration. In some embodiments, the peripheral administration is intravenous injection.

In some aspects, the disclosure provides a method for treating a subject having or suspected of having Parkinson’s disease, the method comprising administering to the subject an isolated nucleic acid, vector, host cell, rAAV, or composition as described herein. In some embodiments, the administration comprises direct injection to the CNS of the subject. In some embodiments, direct injection is intracerebral injection, intraparenchymal injection, intrathecal injection, intra-cisterna magna (ICM) injection or any combination thereof. In some embodiments, the direct injection to the CNS of the subject comprises convection enhanced delivery (CED). In some embodiments, the administration comprises peripheral injection, optionally wherein the peripheral injection is intravenous injection.

In some embodiments, the subject comprises a mutation in a PRKN gene. In some embodiments, the mutation in PRKN gene comprises a nucleotide substitution, deletion, insertion, or splice site mutation.

In some aspects, the disclosure provides recombinant adeno-associated virus (AAV) vector comprising a nucleic acid comprising, in 5′ to 3′ order: a 5′ AAV ITR; a CMV enhancer; a CBA promoter; a transgene encoding a PRKN protein, wherein the PRKN protein is encoded by the nucleic acid sequence in SEQ ID NO: 2 or 3; a WPRE; a Bovine Growth Hormone polyA signal tail; and a 3′ AAV ITR.

In some aspects, the disclosure provides a recombinant adeno-associated virus (rAAV) comprising an AAV capsid protein; and the rAAV vector comprising a nucleic acid comprising, in 5′ to 3′ order: a 5′ AAV ITR; a CMV enhancer; a CBA promoter; a transgene encoding a PRKN protein, wherein the PRKN protein is encoded by the nucleic acid sequence in SEQ ID NO: 2 or 3; a WPRE; a Bovine Growth Hormone polyA signal tail; and a 3′ AAV ITR.

In some embodiments, an AAV capsid protein is AAV9 capsid protein.

In some aspects, the disclosure provides a plasmid comprising an rAAV vector as described herein.

In some aspects, the disclosure provides a Baculovirus vector comprising the nucleic acid sequence set forth in SEQ ID NO: 2 or 3.

In some aspects, the disclosure provides a cell comprising a first vector encoding one or more adeno-associated virus rep protein and/or one or more adeno-associated virus cap protein; and a second vector comprising the nucleic acid sequence set forth in SEQ ID NO: 2 or 3.

In some embodiments, a first vector is a plasmid and a second vector is a plasmid. In some embodiments, a cell is a mammalian cell. In some embodiments, a mammalian cell is a HEK293 cell.

In some embodiments, a first vector is a Baculovirus vector and a second vector is a Baculovirus vector. In some embodiments, a cell is an insect cell. In some embodiments, an insect cell is a SF9 cell.

In some aspects, the disclosure provides a method of producing an rAAV, the method comprising delivering to a cell a first vector encoding one or more adeno-associated virus rep protein and/or one or more adeno-associated cap protein, and a recombinant AAV vector comprising the nucleotide sequence of SEQ ID NO: 2 or 3; culturing the cells under conditions allowing for packaging the rAAV; and harvesting the cultured host cell or culture medium for collection of the rAAV.

In some aspects, the disclosure provides a method for treating a subject having or suspected of having Parkinson’s disease, the method comprising administering to the subject the an rAAV as described herein.

In some embodiments, administration comprises direct injection to the CNS of a subject. In some embodiments, direct injection is intracerebral injection, intraparenchymal injection, intrathecal injection, intra-cisterna magna injection or any combination thereof. In some embodiments, direct injection to the CNS of the subject comprises convection enhanced delivery (CED). In some embodiments, administration comprises peripheral injection. In some embodiments, peripheral injection is intravenous injection.

In some embodiments, a subject is a non-human mammal. In some embodiments, a subject is a human subject.

In some aspects, the disclosure provides a method for correcting mitochondrial dysfunction in a cell, wherein the method comprises contacting the cell with an isolated nucleic acid, a vector or a rAAV as described herein. In some embodiments, the contacting comprises contacting the cell with an amount of the isolated nucleic acid, vector, or rAAV in an amount sufficient to reduce oxidative stress in the cell and/or increase mitophagy in the cell. In some embodiments, the cell is a mammalian cell. In some embodiments, the cell is a human cell. In some embodiments, the cell comprises one or more mutations, insertions, or deletions in a PRKN gene. In some embodiments, the cell is a human cell is in vitro. In some embodiments, the cell is in a subject. In some embodiments, the step of contacting the cell in a subject is by administering to the subject an isolated nucleic acid, a vector, or an rAAV as described herein via any suitable route (e.g., one or more of the routes of administration described herein). In some embodiments, after the contacting occurs, mitochondrial dysfunction is reduced in the cell by at least 1% (e.g., at least 5%, at least 10%, 10-25%, 25-50%, 50-75%, 75-90%, or more than 90%) relative to the mitochondrial dysfunction in the cell prior to the contacting.

### BRIEF DESCRIPTION OF THE FIGURES

FIG. 1 shows a schematic depicting one embodiment of a plasmid comprising a human Parkin-encoding rAAV vector as described by the disclosure.

FIGS. 2A-2B show mRNA and protein expression levels in HeLa cells transfected with codon-optimized Parkin nucleic acid vectors optParkA and optParkB. FIG. 2A shows mRNA expression. FIG. 2B shows optParkA and opt ParkB protein expression in transfected cells.

FIG. 3 shows optParkB protein localization in transfected HeLa cells.

FIGS. 4A-4B show the mitochondrial stress assay on HeLa cells transfected with optParkB. FIG. 4A shows a schematic of the assay. FIG. 4B shows the results of the in vitro assay after menadione dosage.

### DETAILED DESCRIPTION

The disclosure is based, in part, on compositions and methods for expression of one or more gene products (e.g., gene products associated with CNS disease) in a subject. A gene product can be a protein, a fragment (e.g., portion) of a protein, an interfering nucleic acid that inhibits a CNS disease-associated gene, etc. In some embodiments, a gene product is a protein or a protein fragment encoded by a CNS disease-associated gene. In some embodiments, a gene product is an interfering nucleic acid (e.g., shRNA, siRNA, miRNA, amiRNA, etc.) that inhibits a CNS disease-associated gene.

A CNS disease-associated gene refers to a gene encoding a gene product that is genetically, biochemically or functionally associated with a CNS disease, for example Parkinson’s disease (PD). For example, individuals having mutations in a GBA1 gene (GBA1, which encodes the protein Gcase), or a Parkin gene (PRKN, which encodes Parkin protein) have been observed to be have an increased risk of developing PD compared to individuals that do not have a mutation in GBA1 or PRKN. In some embodiments, an expression cassette described herein encodes a wild-type or non-mutant form of a PD-associated gene (or coding sequence thereof) (e.g., Parkin protein). In some embodiments, an expression cassette described herein encodes a wild-type PRKN protein and one or more additional PD-associated proteins. Examples of PD-associated genes are listed in Table 1.

TABLE 1

Examples of PD-associated genes

| | | | |
|---|---|---|---|
| Name | Gene | Function | NCBI Accession No. |
| Parkin | PRKN | plays a neuroprotective role dependent on its Ubiquitin E3 ligase function | BAA25751 (SEQ ID NO: 6) |
| alpha-Synuclein | SNCA | plays a role in maintaining a supply of synaptic vesicles in presynaptic terminals by clustering synaptic vesicles, and may help regulate the release of dopamine | NP_001139527.1 (SEQ ID NO: 7) |
| beta-Glucocerebrosidase | GBA1 | cleaves the beta-glucosidic linkage of glucocerebroside | NP_001005742.1 (Isoform 1(SEQ ID NO: 8)), NP_001165282.1 (Isoform 2 (SEQ ID NO: 9)), NP_001165283.1 (Isoform 3 (SEQ ID NO: 10)) |
| Transmembrane protein 106B | TMEM106B | plays a role in dendrite morphogenesis and regulation of lysosomal trafficking | NP_060844.2 (SEQ ID NO: 11) |
| Progranulin | PGRN | plays a role in development, inflammation, cell proliferation and protein homeostasis | NP_002087.1 (SEQ ID NO: 12) |
| Ribosomal protein S25 | RPS25 | ribosomal protein that is a component of the 40S subunit | AB061844.1 (SEQ ID NO: 13) |
| Microtubule-associated protein tau | MAPT | Microtubule stabilization | NM_016835.4 (SEQ ID NO: 14) |

### Isolated Nucleic Acids and Vectors

An isolated nucleic acid may be DNA or RNA. The disclosure provides, in some aspects, isolated nucleic acids (e.g., rAAV vectors) comprising an expression construct encoding one or more PD-associated genes, for example a Parkin protein (e.g., the gene product of a PRKN gene). “Parkin protein” is an E3 ubiquitin ligase capable of ubiquitinating a wide variety of proteins in response to a variety of conditions (e.g., depolarization of mitochondria or epidermal growth factor signaling). In humans, PRKN gene is located on chromosome 6. In some embodiments, the human PRKN gene encodes a peptide that is represented by NCBI Reference Sequence BAA25751 (SEQ ID NO: 1). In some embodiments an isolated nucleic acid comprises a human Parkin-encoding sequence that has been codon-optimized. In some embodiments, an isolated nucleic acid comprises the codon-optimized sequence set forth in SEQ ID NO: 2 or 3. In some embodiments, an isolated nucleic acid does not comprise the nucleic acid sequence set forth in SEQ ID NO: 4 (e.g., wild type PRKN).

A gene product may be encoded by a coding portion (e.g., a cDNA) of a naturally occurring gene or by a variant of a naturally occurring gene (e.g., a mutant or a truncated version of a naturally occurring gene). In some embodiments, a gene product is a protein (or a fragment thereof) encoded by a human PRKN gene. In some embodiments, a gene product is a protein (or a fragment thereof) encoded by another gene listed in Table 1, for example the MAPT gene. In some embodiments, a gene product is a fragment (e.g., portion) of a gene listed in Table 1, such as a fragment of a human PRKN gene. A protein fragment may comprise about 50%, about 60%, about 70%, about 80% about 90% or about 99% of a protein encoded by the genes listed in Table 1. In some embodiments, a protein fragment comprises between 50% and 99.9% (e.g., any value between 50% and 99.9%) of a protein encoded by a gene listed in Table 1.

An expression construct may comprise one or more promoters (e.g., 1, 2, 3, 4, 5, or more promoters). Any suitable promoter can be used, for example, a constitutive promoter, an inducible promoter, an endogenous promoter, a tissue-specific promoter (e.g., a CNS- specific promoter), etc. In some embodiments, a promoter is a chicken beta-actin promoter (CBA promoter), a CAG promoter (for example as described by Alexopoulou et al. (2008) BMC Cell Biol. 9:2; doi: 10.1186/1471-2121-9-2), or a JeT promoter (for example as described by Tornøe et al. (2002) Gene 297(1-2):21-32).

Aspects of the disclosure relate to constructs which are configured to express one or more transgenes in CNS cells (e.g., neurons or non-neuron cells) of a subject. Thus, in some embodiments, a construct (e.g., gene expression vector) comprises a protein coding sequence that is operably linked to a neuron-specific promoter. Examples of neuron-specific promoters include synapsin I promoter, calcium/calmodulin-dependent protein kinase II promoter, tubulin alpha I promoter, neuron-specific enolase promoter, and platelet-derived growth factor-beta chain promoter, for example as described in Hioki et al. Gene Therapy volume 14, pages872-882(2007).

In some embodiments, an expression construct is monocistronic (e.g., the expression construct encodes a single gene product, for example a protein, or multiple gene products under the control of a single promoter). In some embodiments, an expression construct is polycistronic (e.g., the expression construct encodes two distinct gene products, for example two different proteins or protein fragments, each under the control of a different promoter). A polycistronic expression vector may comprise a one or more (e.g., 1, 2, 3, 4, 5, or more) promoters.

In some embodiments, an expression cassette comprises one or more additional regulatory sequences, including but not limited to transcription factor binding sequences, intron splice sites, poly(A) addition sites, enhancer sequences, repressor binding sites, or any combination of the foregoing. In the context of an expression cassette encoding multiple gene products, a nucleic acid sequence may encode a first gene product and a second gene product, which are separated by a nucleic acid sequence encoding an internal ribosomal entry site (IRES). Examples of IRES sites are described, for example, by Mokrejs et al. (2006) Nucleic Acids Res. 34(Database issue):D125-30. In some embodiments, a nucleic acid sequence encoding a first gene product and a nucleic acid sequence encoding a second gene product are separated by a nucleic acid sequence encoding a self-cleaving peptide. Examples of self-cleaving peptides include but are not limited to T2A, P2A, E2A, F2A, BmCPV 2A, and BmIFV 2A, and those described by Liu et al. (2017) Sci Rep. 7: 2193. In some embodiments, the self-cleaving peptide is a T2A peptide.

Pathologically, disorders such as PD and Gaucher disease are associated with accumulation of protein aggregates composed largely of α-Synuclein (α-Syn) protein. Accordingly, in some embodiments, isolated nucleic acids described herein comprise an inhibitory nucleic acid that reduces or prevents expression of α-Syn protein. A sequence encoding an inhibitory nucleic acid may be placed in an untranslated region (e.g., intron, 5′UTR, 3′UTR, etc.) of the expression vector.

In some embodiments, an inhibitory nucleic acid is positioned in an intron of an expression construct, for example in an intron upstream of the sequence encoding a first gene product. An inhibitory nucleic acid can be a double stranded RNA (dsRNA), siRNA, micro RNA (miRNA), artificial miRNA (amiRNA), or an RNA aptamer. Generally, an inhibitory nucleic acid binds to (e.g., hybridizes with) between about 6 and about 30 (e.g., any integer between 6 and 30, inclusive) contiguous nucleotides of a target RNA (e.g., mRNA). In some embodiments, the inhibitory nucleic acid molecule is an miRNA or an amiRNA, for example an miRNA that targets SNCA (the gene encoding α-Synuclein protein), MAPT (e.g., the gene encoding Tau protein), or APP (e.g., the gene encoding amyloid-beta protein). In some embodiments, the miRNA does not comprise any mismatches with the region of SNCA mRNA, MAPT mRNA, or APP mRNA to which it hybridizes (e.g., the miRNA is “perfected”). In some embodiments, the inhibitory nucleic acid is an shRNA (e.g., an shRNA targeting SNCA, MAPT, or APP). In some embodiments, an inhibitory nucleic acid is an artificial miRNA (amiRNA) that includes a miR-155 scaffold and a SNCA or TMEM106B targeting sequence.

In some embodiments, an inhibitory nucleic acid is an artificial microRNA (amiRNA). A microRNA (miRNA) typically refers to a small, non-coding RNA found in plants and animals and functions in transcriptional and post-translational regulation of gene expression. MiRNAs are transcribed by RNA polymerase to form a hairpin-loop structure referred to as a pri-miRNAs which are subsequently processed by enzymes (e.g., Drosha, Pasha, spliceosome, etc.) to for a pre-miRNA hairpin structure which is then processed by Dicer to form a miRNA/miRNA* duplex (where * indicates the passenger strand of the miRNA duplex), one strand of which is then incorporated into an RNA-induced silencing complex (RISC). In some embodiments, an inhibitory RNA as described herein is a miRNA targeting SNCA, MAPT, or APP.

An artificial microRNA (amiRNA) is derived by modifying native miRNA to replace natural targeting regions of pre-mRNA with a targeting region of interest. For example, a naturally occurring, expressed miRNA can be used as a scaffold or backbone (e.g., a pri-miRNA scaffold), with the stem sequence replaced by that of an miRNA targeting a gene of interest. An artificial precursor microRNA (pre-amiRNA) is normally processed such that one single stable small RNA is preferentially generated. In some embodiments, scAAV vectors and scAAVs described herein comprise a nucleic acid encoding an amiRNA. In some embodiments, the pri-miRNA scaffold of the amiRNA is derived from a pri-miRNA selected from the group consisting of pri-MIR-21, pri-MIR-22, pri-MIR-26a, pri-MIR-30a, pri-MIR-33, pri-MIR-122, pri-MIR-375, pri-MIR-199, pri-MIR-99, pri-MIR-194, pri-MIR-155, pri-MIR-451, pri-MIR-14, pri-MIR145, pri-MIR 7-2 and pri-MIR-155. In some embodiments, an amiRNA comprises a nucleic acid sequence targeting SNCA, MAPT, or APP and an eSIBR amiRNA scaffold, for example as described in Fowler et al. Nucleic Acids Res. 2016 Mar 18; 44(5): e48.

An isolated nucleic acid as described herein may exist on its own, or as part of a vector. Generally, a vector can be a plasmid, cosmid, phagemid, bacterial artificial chromosome (BAC), or a viral vector (e.g., adenoviral vector, adeno-associated virus (AAV) vector, retroviral vector, baculoviral vector, etc.). In some embodiments, the vector is a plasmid (e.g., a plasmid comprising an isolated nucleic acid as described herein). In some embodiments, an rAAV vector is single-stranded (e.g., single-stranded DNA). In some embodiments, the vector is a recombinant AAV (rAAV) vector. In some embodiments, a vector is a Baculovirus vector (e.g., an Autographa californica nuclear polyhedrosis (AcNPV) vector).

Typically, an rAAV vector (e.g., rAAV genome) comprises a transgene (e.g., an expression construct comprising one or more of each of the following: promoter, intron, enhancer sequence, protein coding sequence, inhibitory RNA coding sequence, polyA tail sequence, etc.) flanked by two AAV inverted terminal repeat (ITR) sequences. In some embodiments the transgene of an rAAV vector comprises an isolated nucleic acid as described by the disclosure. In some embodiments, each of the two ITR sequences of an rAAV vector is a full-length ITR (e.g., approximately 145 bp in length, and containing functional Rep binding site (RBS) and terminal resolution site (trs)). In some embodiments, the AAV ITRs are selected from the group consisting of AAV1 ITR, AAV2 ITR, AAV3 ITR, AAV4 ITR, AAV5 ITR, and AAV6 ITR. In some embodiments, one of the ITRs of an rAAV vector is truncated (e.g., shortened or not full-length). In some embodiments, a truncated ITR lacks a functional terminal resolution site (trs) and is used for production of self-complementary AAV vectors (scAAV vectors). In some embodiments, a truncated ITR is a ΔITR, for example as described by McCarty et al. (2003) Gene Ther. 10(26):2112-8.

Aspects of the disclosure relate to isolated nucleic acids (e.g., rAAV vectors) comprising an ITR having one or more modifications (e.g., nucleic acid additions, deletions, substitutions, etc.) relative to a wild-type AAV ITR, for example relative to wild-type AAV2 ITR (e.g., SEQ ID NO: 5). Generally, a wild-type ITR comprises a 125 nucleotide region that self-anneals to form a palindromic double-stranded T-shaped, hairpin structure consisting of two cross arms (formed by sequences referred to as B/B′ and C/C′, respectively), a longer stem region (formed by sequences A/A′), and a single-stranded terminal region referred to as the “D” region. Generally, the “D” region of an ITR is positioned between the stem region formed by the A/A′ sequences and the insert containing the transgene of the rAAV vector (e.g., positioned on the “inside” of the ITR relative to the terminus of the ITR or proximal to the transgene insert or expression construct of the rAAV vector).

An isolated nucleic acid or rAAV vector as described by the disclosure may further comprise a “TRY” sequence, for example as described by Francois, et al. 2005. The Cellular TATA Binding Protein Is Required for Rep-Dependent Replication of a Minimal Adeno-Associated Virus Type 2 p5 Element. J Virol. In some embodiments, a TRY sequence is positioned between an ITR (e.g. a 5′ ITR) and an expression construct (e.g. a transgene-encoding insert) of an isolated nucleic acid or rAAV vector.

In some aspects, the disclosure relates to Baculovirus vectors comprising an isolated nucleic acid or rAAV vector as described by the disclosure. In some embodiments, the Baculovirus vector is an Autographa californica nuclear polyhedrosis (AcNPV) vector, for example as described by Urabe et al. (2002) Hum Gene Ther 13(16):1935-43 and Smith et al. (2009) Mol Ther 17(11):1888-1896.

In some aspects, the disclosure provides a host cell comprising an isolated nucleic acid or vector as described herein. A host cell can be a prokaryotic cell or a eukaryotic cell. For example, a host cell can be a mammalian cell, bacterial cell, yeast cell, insect cell, etc. In some embodiments, a host cell is a mammalian cell, for example a HEK293T cell. In some embodiments, a host cell is a bacterial cell, for example an E. coli cell. In some embodiments, a host cell is an insect cell, for example an SF9 cell (e.g., a clonal isolate of Spodoptera frugiperda Sf21 cells).

### rAAVs

In some aspects, the disclosure relates to recombinant AAVs (rAAVs) comprising a transgene that encodes a nucleic acid as described herein (e.g., an rAAV vector as described herein). The term “rAAVs” generally refers to viral particles comprising an rAAV vector encapsidated by one or more AAV capsid proteins. In some embodiments, an rAAV is a self-complementary rAAV (scAAV).

An rAAV described by the disclosure may comprise a capsid protein having a serotype selected from AAV1, AAV2, AAV3, AAV4, AAV5, AAV6, AAV7, AAV8, AAV9, AAV10, and variants thereof. In some embodiments, an rAAV comprises a capsid protein having a serotype of AAV9. In some embodiments, an rAAV comprises a capsid protein from a non-human host, for example a rhesus AAV capsid protein such as AAVrh.10, AAVrh.39, etc. In some embodiments, an rAAV described by the disclosure comprises a capsid protein that is a variant of a wild-type capsid protein, such as a capsid protein variant that includes at least 1, 2, 3, 4, 5, 6, 7, 8, 9, 10, or more than 10 (e.g., 15, 20 25, 50, 100, etc.) amino acid substitutions (e.g., mutations) relative to the wild-type AAV capsid protein from which it is derived. In some embodiments, an rAAV comprises a chimeric capsid protein (e.g., a capsid protein that comprises sequences from two or more AAV different capsid proteins), for example AAV1RX, as described by Albright et al. Mol Ther. 2018 Feb 7;26(2):510-523. In some embodiments, a capsid protein variant is an AAV TM6 capsid protein, for example as described by Rosario et al. Mol Ther Methods Clin Dev. 2016; 3: 16026.

In some embodiments, an rAAV described by the disclosure comprises a capsid protein that is a variant of a wild-type capsid protein, such as a capsid protein variant that includes at least 1, 2, 3, 4, 5, 6, 7, 8, 9, 10, or more than 10 (e.g., 15, 20 25, 50, 100, etc.) amino acid substitutions (e.g., mutations) relative to the wild-type AAV capsid protein from which it is derived. In some embodiments, a capsid protein variant comprises an amino acid sequence that is at least 70%, at least 80%, at least 90%, at least 95%, at least or 99% identical to the wild-type AAV capsid protein from which it is derived.

The disclosure is based, in part, on rAAVs containing a transgene encoding one or more PD-associated gene products (e.g., human Parkin) and capsid proteins which target cells in the central nervous system (CNS), for example neuron cells (e.g., astrocytes) or non-neuronal cells (e.g., microglial cells, perivascular macrophages, choroid plexus macrophages, meningeal macrophages, meningeal dendritic cells, and/or meningeal granulocytes).

In some embodiments, rAAVs described by the disclosure readily spread through the CNS, particularly when introduced into the CSF space or directly into the brain parenchyma. Accordingly, in some embodiments, rAAVs described by the disclosure comprise a capsid protein that is capable of crossing the blood-brain barrier (BBB). For example, in some embodiments, an rAAV comprises a capsid protein having an AAV9 serotype, AAVrh.10 serotype, or AAV1RX serotype. Production of rAAVs is described, for example, by Samulski et al. (1989) J Virol. 63(9):3822-8 and Wright (2009) Hum Gene Ther. 20(7): 698-706.

In some embodiments, an rAAV as described by the disclosure (e.g., comprising a recombinant rAAV genome encapsidated by AAV capsid proteins to form an rAAV capsid particle) is produced in a Baculovirus vector expression system (BEVS). Production of rAAVs using BEVS are described, for example by Urabe et al. (2002) Hum Gene Ther 13(16):1935-43, Smith et al. (2009) Mol Ther 17(11):1888-1896, U.S. Pat. No. 8,945,918, U.S. Pat. No. 9,879,282, and International PCT Publication WO 2017/184879. However, an rAAV can be produced using any suitable method (e.g., using recombinant rep and cap genes).

### Pharmaceutical Compositions

In some aspects, the disclosure provides pharmaceutical compositions comprising an isolated nucleic acid or rAAV as described herein and a pharmaceutically acceptable carrier. As used herein, the term “pharmaceutically acceptable” refers to a material, such as a carrier or diluent, which does not abrogate the biological activity or properties of the compound, and is relatively non-toxic, e.g., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Additional ingredients that may be included in the pharmaceutical compositions used in the practice of the invention are known in the art and described, for example in Remington’s Pharmaceutical Sciences (Genaro, Ed., Mack Publishing Co., 1985, Easton, PA), which is incorporated herein by reference.

Compositions (e.g., pharmaceutical compositions) provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

### Methods

Parkinson’s disease has been associated with mitochondrial dysfunction and oxidative stress in the cells (or cellular environment) of a subject. For example, deficiency in complex I of the mitochondrial electron transport chain has been observed in PD patients. In some embodiments, mitochondrial dysfunction is caused by a mutation in one or more of the following genes in a cell or subject: SNCA, LRRK2, PRKN, PINK1 or ATP13A2. PRKN encodes a cytosolic E3 ubiquitin ligase that ubiquitinates target proteins for signaling or proteasomal degradation. In some embodiments, Parkin functions in maintaining healthy mitochondria by regulating their biogenesis and degradation via mitophagy. Certain mutations in PRKN disrupt this process and result in mitochondrial dysregulation and an increase in oxidative stress, for example as described in Park et al. Curr. Neurol Neurosci. Rep. 2018; 18(5): 21.

The disclosure is based, in part, on compositions and methods for reducing mitochondrial dysfunction and/or oxidative stress in a cell or subject. In some embodiments, the disclosure provides a method for reducing mitochondrial dysfunction in a cell or subject (e.g., reducing oxidative stress in a cell or subject) by administering a composition (e.g., a composition comprising an isolated nucleic acid or a vector or a rAAV) as described by the disclosure. In some embodiments, administration of a composition of the disclosure to a subject reduces mitochondrial dysfunction or oxidative stress in the cell or subject by at least 1%, 5%, 10%, 20%, 30%, 40%, 50%, 60%, 70%, 80%, 90%, 99%, 100%, or more (e.g., relative to a subject that is not characterized by mitochondrial dysfunction or oxidative stress, or relative to the level of mitochondrial dysfunction or oxidative stress in the subject prior to administration of the composition). Methods of measuring levels of mitochondrial function and oxidative stress are known in the art. In some embodiments, mitochondrial function is measured by oxygen consumption, luminescent ATP assays for quantification of total energy metabolism, and MTT or Alamar Blue for determination of metabolic activity. In some embodiments, oxidative stress is measured by levels of DNA/RNA damage, lipid peroxidation, and protein oxidation/nitration, or directly measuring reactive oxygen species.

Aspects of the disclosure relate to compositions for expression of one or more CNS disease-associated gene products in a subject to treat CNS-associated diseases. The one or more CNS disease-associated gene products may be encoded by one or more isolated nucleic acids or rAAV vectors. In some embodiments, a subject is administered a single vector (e.g., isolated nucleic acid, rAAV, etc.) encoding one or more (1, 2, 3, 4, 5, or more) gene products. In some embodiments, a subject is administered a plurality (e.g., 2, 3, 4, 5, or more) vectors (e.g., isolated nucleic acids, rAAVs, etc.), where each vector encodes a different CNS disease-associated gene product. In some embodiments, the transgene delivered to the target cell encodes one or more PD-associated proteins, for example human Parkin and/or one or more inhibitory nucleic acids targeting APP, MAPT, or α-Synuclein.

In some aspects, compositions (e.g., isolated nucleic acids, rAAVs, etc.) described herein are administered to a subject. In some embodiments, a subject is a human. In some embodiments, a subject is administered more than one (e.g., 2, 3, 4, 5, or more) vector (e.g., rAAV), each vector encoding a different transgene (e.g., a first rAAV encoding a human Parkin protein, and a second rAAV encoding a GBA1 protein or an inhibitory nucleic acid).

The disclosure is based, in part, on compositions for expression of PD-associated gene products in a subject to treat Parkinson’s disease. As used herein “treat” or “treating” refers to (a) preventing or delaying onset of Parkinson’s disease; (b) reducing severity of Parkinson’s disease; (c) reducing or preventing development of symptoms characteristic of Parkinson’s disease; (d) and/or preventing worsening of symptoms characteristic of Parkinson’s disease. Signs and symptoms of Parkinson’s disease include, for example, accumulation of synuclein protein, tremor, slowed movement (bradykinesia), rigid muscles (stiffness), impaired posture and balance, speech change and writing changes.

The disclosure is based, in part, on compositions for expression of combinations of PD-associated gene products in a subject that act together (e.g., synergistically) to treat Parkinson’s disease.

Accordingly, in some aspects, the disclosure provides a method for treating a subject having or suspected of having Parkinson’s disease, the method comprising administering to the subject a composition (e.g., a composition comprising an isolated nucleic acid or a vector or a rAAV) as described by the disclosure.

The disclosure is based, in part, on compositions for expression of one or more CNS-disease associated gene products in a subject to treat Gaucher disease. In some embodiments, the Gaucher disease is a neuronopathic Gaucher disease, for example Type 2 Gaucher disease or Type 3 Gaucher disease. In some embodiments, a subject does not have PD or PD symptoms.

Accordingly, in some aspects, the disclosure provides a method for treating a subject having or suspected of having neuronopathic Gaucher disease, the method comprising administering to the subject a composition (e.g., a composition comprising an isolated nucleic acid or a vector or a rAAV) as described by the disclosure.

In some embodiments, the disclosure provides a method for treating a subject having or suspected of having autosomal recessive juvenile Parkinson’s disease. In some embodiments, the disclosure provides a method for treating a subject having or suspected of having Parkinson’s Disease with a PARK2 mutation. In some embodiments, the disclosure provides a method for treating a subject having or suspected of having idiopathic Parkinson’s Disease. In some embodiments, the disclosure provides a method for treating a subject having or suspected of having sporadic Parkinson’s Disease. In some embodiments, the disclosure provides a method for treating a subject having or suspected of having atypical Parkinsonism. In some embodiments, the disclosure provides a method for treating a subject having or suspected of having Multiple System Atrophy (MSA). In some embodiments, the disclosure provides a method for treating a subject having or suspected of having Progressive Supranuclear Palsy (PSP). In some embodiments, the disclosure provides a method for treating a subject having or suspected of having Corticobasal Syndrome (CBS). In some embodiments, the disclosure provides a method for treating a subject having or suspected of having Dementia with Lewy bodies (DLB). In some embodiments, the disclosure provides a method for treating a subject having or suspected of having drug-induced Parkinsonism. In some embodiments, the disclosure provides a method for treating a subject having or suspected of having Vascular Parkinsonism (VP).

In some embodiments, a subject having or at risk of developing Parkinson’s disease is characterized by having one or more mutations, substitutions, insertions, or deletions in human PRKN.

Examples of mutations in human PRKN (or human Parkin protein) include c.81G>T, A42P, Exon 2 deletion, Exon 2 duplication, 255delA, 202-3delAG, A46P, Q34R, D53E, Exon 30 40 bp del, Exon 3 deletion, R128K, Exon 3-4 deletion, Exon 5-6 deletion, Exon 6 deletion, T240M, R275W, R256C, I298L, Exon 8 deletion, P437A, 255delA, Exon 3 40 bp deletion, R256C + Exon 2-4 deletion, 255delA+exon 2-4 deletion, 255delA +R275W, R42P + R275W, 202delAG + Exon 3-4 deletion, R42P + exon 3 deletion, Exon 3 40bp deletion, Exon 3 40bp deletion +exon 4 deletion, Exon 3 deletion + Exon 5 deletion, Exon 3 40bp deletion +R275W, Exon 3 deletion +Exon 12 deletion, Exon 3 40bp deletion + G430D, Exon 3-4 deletion, G430D, Exon 4 deletion + R275W, Exon 4 deletion+ R366Q, R275W + G430D, Exon 7-8 duplication, exon 10 deletion, R275W +C212Y, C253W, R256C, R275W, and D280N, exon 7 mutations, or a combination thereof.

A subject is typically a mammal, preferably a human. In some embodiments, a subject is between the ages of 1 month old and 10 years old (e.g., 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years, 5 years, 6 years, 7 years, 8 years, 9 years, 10 years, or any age therebetween). In some embodiments, a subject is between 2 years old and 20 years old. In some embodiments, a subject is between 30 years old and 100 years old. In some embodiments, a subject is older than 55 years old.

In some aspects, the disclosure provides a method for treating a subject having or suspected of having Parkinson’s disease, the method comprising administering to the subject a composition (e.g., a composition comprising an isolated nucleic acid or a vector or a rAAV) as described by the disclosure.

In some aspects, compositions of the disclosure are useful for treating a CNS-associated disease. A CNS-associated disease may be a neurodegenerative disease, synucleinopathy, tauopathy, or a lysosomal storage disease. Examples of neurodegenerative diseases and their associated genes are listed in Table 2.

A “synucleinopathy” refers to a disease or disorder characterized by 1) reduced expression or activity of alpha-Synuclein (the gene product of SNCA) in a subject (e.g., relative to a healthy subject, for example a subject not having a synucleinopathy) or 2) increased expression or activity of alpha-Synuclein (the gene product of SNCA) in a subject (e.g., relative to a healthy subject, for example a subject not having a synucleinopathy) that results in a toxic “gain of function” phenotype. Examples of synucleinopathies and their associated genes are listed in Table 3.

A “tauopathy” refers to a disease or disorder characterized by 1) reduced expression or activity of Tau protein in a subject (e.g., a healthy subject not having a tauopathy) or 2) increased expression or activity of Tau protein in a subject (e.g., a healthy subject not having a tauopathy) that results in a “gain of function” phenotype. Examples of tauopathies and their associated genes are listed in Table 4.

A “lysosomal storage disease” refers to a disease characterized by 1) abnormal build-up of toxic cellular products in lysosomes of a subject or 2) absence of a gene product expressed in lysosomes of a subject that leads to a deficiency in or abnormal build-up of certain cellular products (e.g., lysosomal enzymes, lipids, metabolites, etc.) in lysosomes. Examples of lysosomal storage diseases and their associated genes are listed in Table 5.

In some embodiments, a composition is administered directly to the CNS of the subject, for example by direct injection into the brain and/or spinal cord of the subject. Examples of CNS-direct administration modalities include but are not limited to intracerebral injection, intraventricular injection, intracisternal injection, intraparenchymal injection, intrathecal injection, and any combination of the foregoing. In some embodiments, direct injection into the CNS of a subject results in transgene expression (e.g., expression of a human Parkin protein) in the midbrain, striatum and/or cerebral cortex of the subject. In some embodiments, direct injection into the CNS results in transgene expression (e.g., expression of a human Parkin protein) in the spinal cord and/or CSF of the subject. In some embodiments, direct administration to the CNS of a subject results in infection of CNS cells of the subject with the rAAV. In some embodiments, direct administration to the CNS of a subject results in expression of the transgene encoded by the rAAV (e.g., human PRKN, etc.) in CNS cells of the subject. In some embodiments, the myeloid cells are microglial cells.

In some embodiments, direct injection to the CNS of a subject comprises convection enhanced delivery (CED). Convection enhanced delivery is a therapeutic strategy that involves surgical exposure of the brain and placement of a small-diameter catheter directly into a target area of the brain, followed by infusion of a therapeutic agent (e.g., a composition or rAAV as described herein) directly to the brain of the subject. CED is described, for example by Debinski et al. (2009) Expert Rev Neurother. 9(10):1519-27.

In some embodiments, a composition is administered peripherally to a subject, for example by peripheral injection. Examples of peripheral injection include subcutaneous injection, intravenous injection, intra-arterial injection, intraperitoneal injection, or any combination of the foregoing. In some embodiments, the peripheral injection is intra-arterial injection, for example injection into the carotid artery of a subject.

In some embodiments, a composition (e.g., a composition comprising an isolated nucleic acid or a vector or a rAAV) as described by the disclosure is administered both peripherally and directly to the CNS of a subject. For example, in some embodiments, a subject is administered a composition by intra-arterial injection (e.g., injection into the carotid artery) and by intraparenchymal injection (e.g., intraparenchymal injection by CED). In some embodiments, the direct injection to the CNS and the peripheral injection are simultaneous (e.g., happen at the same time). In some embodiments, the direct injection occurs prior (e.g., between 1 minute and 1 week, or more before) to the peripheral injection. In some embodiments, the direct injection occurs after (e.g., between 1 minute and 1 week, or more after) the peripheral injection.

The amount of composition (e.g., a composition comprising an isolated nucleic acid or a vector or a rAAV) as described by the disclosure administered to a subject will vary depending on the administration method. For example, in some embodiments, a rAAV as described herein is administered to a subject at a titer between about 10⁹ Genome copies (GC)/kg and about 10¹⁴ GC/kg (e.g., about 10⁹ GC/kg, about 10¹⁰ GC/kg, about 10¹¹ GC/kg, about 10¹² GC/kg, about 10¹² GC/kg, or about 10¹⁴ GC/kg). In some embodiments, a subject is administered a high titer (e.g., >10¹² Genome Copies GC/kg of an rAAV) by injection to the CSF space, or by intraparenchymal injection.

A composition (e.g., a composition comprising an isolated nucleic acid or a vector or a rAAV) as described by the disclosure can be administered to a subject once or multiple times (e.g., 2, 3, 4, 5, 6, 7, 8, 9, 10, 20, or more) times. In some embodiments, a composition is administered to a subject continuously (e.g., chronically), for example via an infusion pump.

TABLE 2

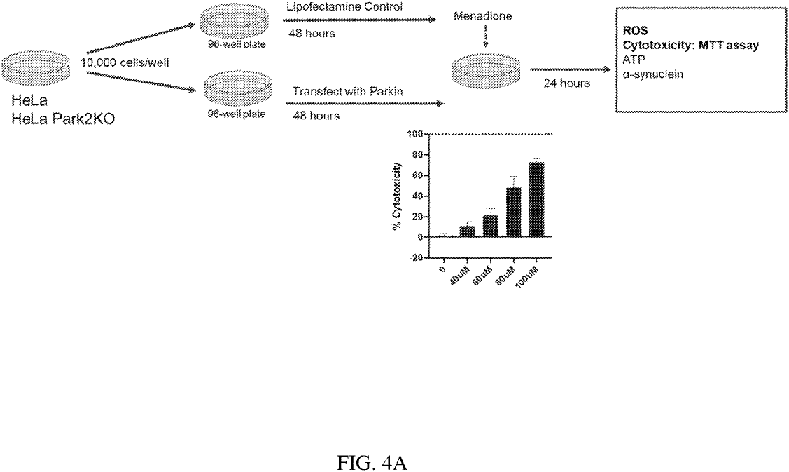

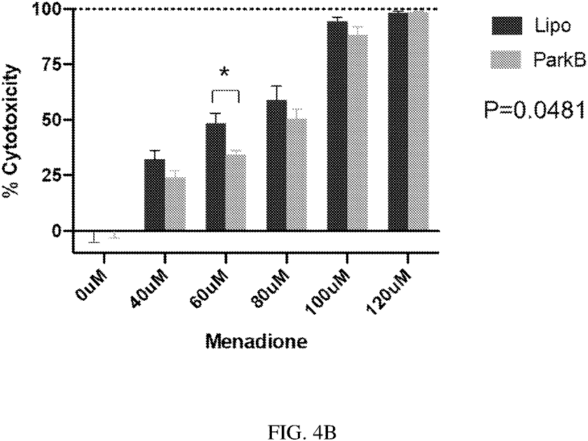

Examples of neurodegenerative diseases

| | |
|---|---|
| Disease | Associated genes |
| Alzheimer’s disease | APP, PSEN1, PSEN2, APOE |
| Parkinson’s disease | LRRK2, PARK7, PINK1, PRKN, SNCA, GBA, UCHL1, ATP13A2, VPS35 |
| Huntington’s disease | HTT |
| Amyotrophic lateral sclerosis | ALS2, ANG, ATXN2, C9orf72, CHCHD10, CHMP2B, DCTN1, ERBB4, FIG. 4 , FUS, HNRNPA1, MATR3, NEFH, OPTN, PFN1, PRPH, SETX, SIGMAR1, SMN1, |
| | SOD1, SPG11, SQSTM1, TARDBP, TBK1, TRPM7, TUBA4A, UBQLN2, VAPB, VCP |
| Batten disease (Neuronal ceroid lipofunscinosis) | PPT1, TPP1, CLN3, CLN5, CLN6, MFSD8, CLN8, CTSD, DNAJC5, CTSF, ATP13A2, GRN, KCTD7 |
| Friedreich’s ataxia | FXN |
| Lewy body disease | APOE, GBA, SNCA, SNCB |
| Spinal muscular atrophy | SMN1, SMN2 |
| Multiple sclerosis | CYP27B1, HLA-DRB1, IL2RA, IL7R, TNFRSF1A |
| Prion disease (Creutzfeldt-Jakob disease, Fatal familial insomnia, Gertsmann-Straussler-Scheinker syndrome, Variably protease-sensitive prionopathy) | PRNP |

TABLE 3

Examples of synucleinopathies

| | |
|---|---|
| Disease | Associated genes |
| Parkinson’s disease | LRRK2, PARK7, PINK1, PRKN, SNCA, GBA, UCHL1, ATP13A2, VPS35 |
| Dementia with Lewy bodies | APOE, GBA, SNCA, SNCB |
| Multiple system atrophy | COQ2, SNCA |

TABLE 4

Examples of tauopathies

| | |
|---|---|
| Disease | Associated genes |
| Alzheimer’s disease | APP, PSEN1, PSEN2, APOE |
| Primary age-related tauopathy | MAPT |
| Progressive supranuclear palsy | MAPT |
| Corticobasal degeneration | MAPT, GRN, C9orf72, VCP, CHMP2B, TARDBP, FUS |
| Frontotemporal dementia with parkinsonism-17 | MAPT |
| Subacute sclerosing panencephalitis | SCN1A |
| Lytico-Bodig disease | |
| Gangioglioma, gangliocytoma | |
| Meningioangiomatosis | |
| Postencephalitic parkinsonism | |
| Chronic traumatic encephalopathy | |

TABLE 5

Examples of lysosomal storage diseases

| | |
|---|---|
| Disease | Associated genes |
| Niemann-Pick disease | NPC1, NPC2, SMPD1 |
| Fabry disease | GLA |
| Krabbe disease | GALC |
| Gaucher disease | GBA |
| Tach-Sachs disease | HEXA |
| Metachromatic leukodystrophy | ARSA, PSAP |
| Farber disease | ASAH1 |
| Galactosialidosis | CTSA |
| Schindler disease | NAGA |
| GM1 gangliosidosis | GLB1 |
| GM2 gangliosidosis | GM2A |
| Sandhoff disease | HEXB |
| Lysosomal acid lipase deficiency | LIPA |
| Multiple sulfatase deficiency | SUMF1 |
| Mucopolysaccharidosis Type I | IDUA |
| Mucopolysaccharidosis Type II | IDS |
| Mucopolysaccharidosis Type III | GNS, HGSNAT, NAGLU, SGSH |
| Mucopolysaccharidosis Type IV | GALNS, GLB1 |
| Mucopolysaccharidosis Type VI | ARSB |
| Mucopolysaccharidosis Type VII | GUSB |
| Mucopolysaccharidosis Type IX | HYAL1 |
| Mucolipidosis Type II | GNPTAB |
| Mucolipidosis Type III alpha/beta | GNPTAB |
| Mucolipidosis Type III gamma | GNPTG |
| Mucolipidosis Type IV | MCOLN1 |
| Neuronal ceroid lipofuscinosis | PPT1, TPP1, CLN3, CLN5, CLN6, MFSDS8, CLN8, CTSD, DNAJC5, CTSF, ATP13A2, GRN, KCTD7 |
| Alpha-mannosidosis | MAN2B1 |
| Beta-mannosidosis | MANBA |
| Aspartylglucosaminuria | AGA |
| Fucosidosis | FUCA1 |

### EXAMPLES

### Example 1: rAAV Vectors

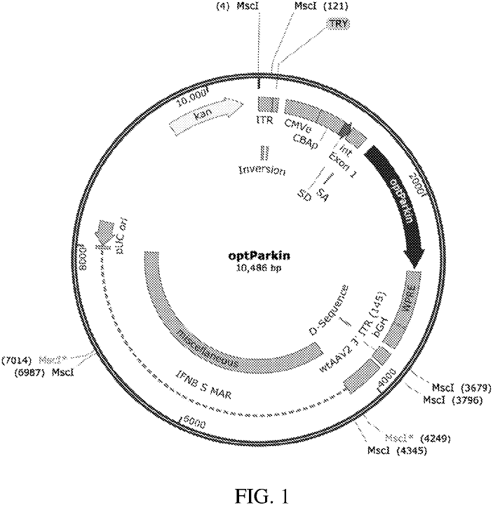

AAV vectors are generated using cells, such as HEK293 cells for triple-plasmid transfection or SF9 cells for Baculovirus-based production. The ITR sequences flank an expression construct comprising a promoter/enhancer element operably linked to a codon-optimized nucleic acid sequence encoding human PRKN protein (e.g., SEQ ID NO: 2 or 3), a 3′ polyA signal, and posttranslational signals such as the WPRE element. FIG. 1 shows one embodiment of a plasmid encoding an rAAV vector encoding human Parkin.

### Example 2: Assays

In some embodiments, the expression and/or activity of a protein (e.g., a PRKN protein) encoded by a nucleic acid composition (e.g., one or more nucleic acids described throughout this application) can be evaluated using one or more assays. The following paragraphs provide non-limiting examples of assays that can be used to evaluate nucleic acid and/or protein expression and/or activity.

### A. mRNA and Protein Expression Assays

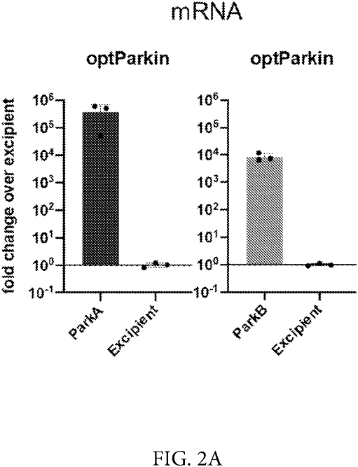

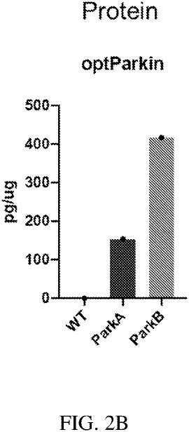

HeLa cells were transfected with 50 ng plasmid DNA/well of plasmids L00310 (comprising SEQ ID NO: 2, also referred to as optParkA) and L00311 (comprising SEQ ID NO: 3, also referred to as optParkB) using Lipofectamine 2000. Cells were incubated at 37° C. for 72 hours. mRNA and protein expression of optParkA and optParkB were measured. For mRNA expression measurement, cells were lysed and cDNA was made using cells-to-ct kit. qRT-PCR assay was done using SYBR green (FIG. 2A). Reverse primers for the qRT-PCR assay were different for optParkA and optParkB. For protein expression measurement, cells were lysed and assayed using Abcam human Parkin Simple Step ELISA kit (FIG. 2B). Results show that optParkB protein expression is more than twice that of optParkA.

### B. Protein Localization Assay

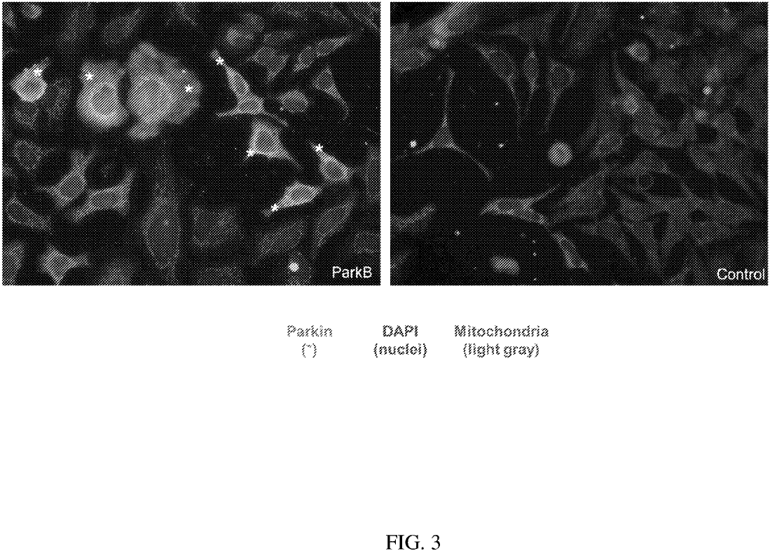

HeLa cells were transfected with 100 ng L00311(optParkB) using Lipofectamine 2000 and incubated for 72 hours at 37° C. Cells were stained for mitochondria, fixed and stained for nucleus and Parkin. Results in FIG. 3 show stained cell nuclei as large rounded areas in the center of cells, and mitochondria in light gray. The optParkB protein (darker gray areas in the cytoplasm, indicated by asterisks “*” in FIG. 3) is localized in the cytoplasm of transfected cells.

### C. Mitochondrial Stress Assay

HeLa cells were transfected with 10 ng per well L00311 (optParkB) using Lipofectamine 2000 and incubated for 48 hours at 37° C. Cells were dosed with Menadione at 48 hours and MTT assay was completed at 72 hours post-transfection (FIG. 4A). Results show that HeLa cells transfected with 10 ng Parkin have less cytotoxicity at a 60 uM Menadione dose (FIG. 4B). No significant difference was observed with 5 ng or 20 ng Parkin transfections. Cell toxicity was observed in 50 ng Parkin transfection.

### EQUIVALENTS

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one step or act, the order of the steps or acts of the method is not necessarily limited to the order in which the steps or acts of the method are recited.

### SEQUENCES

In some embodiments, an expression cassette encoding one or more gene products (e.g., a first, second and/or third gene product) comprises or consists of (or encodes a peptide having) a sequence set forth in any one of SEQ ID NOs: 1-5. In some embodiments, a gene product comprises or consists of or is encoded by a portion (e.g., fragment) of any one of SEQ ID NOs: 1-5. >human Parkin amino acid sequence (SEQ ID NO: 1)

MIVFVRFNSSHGFPVEVDSDTSIFQLKEVVAKRQGVPADQLRVIFAGKEL
RNDWTVQNCDLDQQSIVHIVQRPWRKGQEMNATGGDDPRNAAGGCEREPQ
SLTRVDLSSSVLPGDSVGLAVILHTDSRKDSPPAGSPAGRSIYNSFYVYC
KGPCQRVQPGKLRVQCSTCRQATLTLTQGPSCWDDVLIPNRMSGECQSPH
CPGTSAEFFFKCGAHPTSDKETSVALHLIATNSRNITCITCTDVRSPVLV
FQCNSRHVICLDCFHLYCVTRLNDRQFVHDPQLGYSLPCVAGCPNSLIKE
LHHFRILGEEQYNRYQQYGAEECVLQMGGVLCPRPGCGAGLLPEPDQRKV
TCEGGNGLGCGFAFCRECKEAYHEGECSAVFEASGTTTQAYRVDERAAEQ
ARWEAASKETIKKTTKPCPRCHVPVEKNGGCMHMKCPQPQCRLEWCWNCG
CEWNRVCMGDHWFDV

>human Parkin nucleic acid sequence; codon-optimized (SEQ ID NO: 2)

ATGATTGTTTTCGTCAGATTCAATAGTTCCCACGGGTTCCCTGTCGAGGT
GGACAGTGATACTAGCATCTTCCAGCTGAAAGAAGTGGTGGCGAAGCGGC
AGGGAGTTCCTGCAGACCAACTGAGGGTCATTTTCGCCGGCAAGGAGCTG
AGGAACGATTGGACTGTGCAGAACTGTGACCTTGATCAGCAGAGTATCGT
TCACATAGTGCAGCGCCCGTGGAGGAAGGGGCAGGAGATGAACGCAACGG
GCGGGGACGACCCCAGAAATGCTGCGGGGGGTTGCGAGCGGGAACCTCAG
TCTCTGACTCGGGTGGACCTGTCTAGCTCTGTGCTCCCAGGTGATAGCGT
TGGCCTCGCTGTTATCCTGCATACAGACTCCAGGAAGGATAGTCCTCCCG
CCGGGTCTCCTGCCGGCCGAAGTATCTATAACTCATTTTACGTTTACTGC
AAAGGACCCTGCCAACGCGTACAACCCGGCAAGCTCCGCGTGCAATGCTC
AACTTGTAGGCAGGCCACACTCACTTTGACGCAAGGTCCCTCTTGCTGGG
ACGATGTGCTGATTCCGAATAGAATGAGTGGCGAGTGCCAATCACCCCAT
TGTCCCGGTACAAGCGCGGAATTCTTCTTCAAATGCGGCGCACATCCCAC
GTCAGACAAAGAGACTTCAGTCGCTCTCCACCTGATAGCCACCAACTCCC
GCAACATTACCTGTATAACTTGCACGGATGTCCGCTCCCCCGTGTTGGTG
TTCCAGTGTAACTCCAGACATGTGATCTGTCTGGACTGCTTTCACCTGTA
CTGCGTGACTAGACTTAATGACAGACAGTTTGTACATGACCCCCAGCTGG
GATACAGCCTGCCGTGCGTGGCCGGTTGTCCCAACAGCCTGATTAAGGAG
CTGCACCATTTCAGGATCCTGGGCGAGGAGCAGTACAACAGATACCAGCA
GTACGGGGCGGAGGAGTGTGTTCTTCAGATGGGGGGGGTGCTGTGCCCCA
GGCCCGGCTGCGGTGCTGGTCTGTTGCCAGAGCCCGACCAGAGAAAGGTC
ACATGTGAGGGCGGTAATGGGCTTGGCTGTGGATTTGCCTTCTGCAGGGA
ATGTAAAGAGGCCTACCACGAGGGCGAATGCAGTGCCGTTTTCGAAGCAA
GTGGCACCACAACACAGGCCTATAGAGTTGATGAAAGGGCAGCAGAACAA
GCGAGGTGGGAAGCCGCCTCCAAAGAAACTATCAAAAAAACGACAAAGCC
ATGCCCCAGGTGCCATGTGCCTGTGGAGAAAAACGGGGGATGCATGCATA
TGAAATGTCCCCAGCCCCAGTGCCGGTTGGAGTGGTGTTGGAACTGTGGC
TGCGAATGGAATCGGGTCTGCATGGGGGACCACTGGTTTGACGTG

>human Parkin nucleic acid sequence; codon-optimized (SEQ ID NO: 3)

ATGATTGTGTTCGTTCGATTCAATTCATCCCATGGATTTCCAGTCGAGGT
CGATTCAGATACCTCCATATTCCAGCTCAAAGAAGTTGTCGCAAAGAGGC
AAGGAGTGCCAGCCGACCAGCTGCGAGTCATCTTTGCCGGAAAGGAGTTG
AGGAACGACTGGACCGTTCAAAATTGTGACCTGGACCAGCAGTCAATAGT
GCACATCGTGCAAAGGCCTTGGCGGAAGGGTCAAGAGATGAACGCTACTG
GTGGCGACGATCCTCGGAATGCAGCAGGCGGCTGCGAACGAGAGCCTCAG
AGCCTTACCAGGGTAGATTTGTCATCCAGCGTATTGCCTGGTGACTCAGT
AGGACTGGCTGTAATTCTTCATACAGACAGCAGGAAAGATAGCCCACCAG
CCGGCAGCCCCGCTGGTAGAAGTATCTACAACTCATTCTACGTCTATTGC
AAAGGGCCGTGTCAGCGGGTGCAACCGGGTAAACTCAGAGTCCAATGCAG
CACCTGTAGACAAGCTACACTGACACTTACACAAGGGCCTAGTTGTTGGG
ACGACGTTCTTATTCCCAATAGAATGTCAGGTGAGTGTCAAAGTCCTCAT
TGTCCGGGGACTAGTGCTGAGTTTTTTTTCAAATGCGGCGCTCACCCCAC
TAGTGACAAGGAGACAAGCGTGGCCCTGCATCTCATAGCGACGAATAGCA
GAAACATAACATGCATCACTTGCACGGACGTTCGGTCACCTGTGCTTGTG
TTTCAATGTAACAGCCGGCATGTCATTTGTCTTGATTGCTTTCACCTCTA
CTGTGTGACACGCTTGAATGACAGACAATTCGTCCATGACCCACAATTGG
GATACAGTTTGCCCTGCGTAGCGGGTTGTCCAAATTCTTTGATTAAGGAG
CTGCATCACTTTCGGATCCTGGGAGAAGAGCAGTACAATCGATACCAGCA
GTATGGAGCTGAAGAGTGTGTGCTCCAAATGGGCGGGGTTCTTTGTCCCC
GGCCTGGCTGCGGCGCCGGTTTGCTCCCCGAACCAGATCAGCGGAAAGTT
ACATGTGAGGGTGGAAATGGTCTTGGCTGTGGCTTCGCGTTCTGCCGGGA
GTGCAAAGAAGCGTACCATGAAGGGGAGTGCAGCGCAGTTTTTGAGGCAA
GTGGCACGACGACCCAGGCTTACCGGGTAGACGAACGCGCAGCAGAGCAG
GCCAGATGGGAAGCGGCCTCCAAGGAGACCATTAAAAAGACAACCAAACC
TTGTCCTCGGTGTCACGTGCCCGTCGAGAAGAACGGGGGCTGTATGCATA
TGAAATGCCCACAACCGCAATGTAGGCTGGAATGGTGTTGGAACTGCGGC
TGCGAATGGAATAGGGTGTGTATGGGAGACCATTGGTTTGACGTCTAG

>human Parkin wild type nucleic acid sequence (SEQ ID NO: 4)

ATGATAGTGTTTGTCAGGTTCAACTCCAGCCATGGTTTCCCAGTGGAGGT
CGATTCTGACACCAGCATCTTCCAGCTCAAGGAGGTGGTTGCTAAGCGAC
AGGGGGTTCCGGCTGACCAGTTGCGTGTGATTTTCGCAGGGAAGGAGCTG
AGGAATGACTGGACTGTGCAGAATTGTGACCTGGATCAGCAGAGCATTGT
TCACATTGTGCAGAGACCGTGGAGAAAAGGTCAAGAAATGAATGCAACTG
GAGGCGACGACCCCAGAAACGCGGCGGGAGGCTGTGAGCGGGAGCCCCAG
AGCTTGACTCGGGTGGACCTCAGCAGCTCAGTCCTCCCAGGAGACTCTGT
GGGGCTGGCTGTCATTCTGCACACTGACAGCAGGAAGGACTCACCACCAG
CTGGAAGTCCAGCAGGTAGATCAATCTACAACAGCTTTTATGTGTATTGC
AAAGGCCCCTGTCAAAGAGTGCAGCCGGGAAAACTCAGGGTACAGTGCAG
CACCTGCAGGCAGGCAACGCTCACCTTGACCCAGGGTCCATCTTGCTGGG
ATGATGTTTTAATTCCAAACCGGATGAGTGGTGAATGCCAATCCCCACAC
TGCCCTGGGACTAGTGCAGAATTTTTCTTTAAATGTGGAGCACACCCCAC
CTCTGACAAGGAAACATCAGTAGCTTTGCACCTGATCGCAACAAATAGTC
GGAACATCACTTGCATTACGTGCACAGACGTCAGGAGCCCCGTCCTGGTT
TTCCAGTGCAACTCCCGCCACGTGATTTGCTTAGACTGTTTCCACTTATA
CTGTGTGACAAGACTCAATGATCGGCAGTTTGTTCACGACCCTCAACTTG
GCTACTCCCTGCCTTGTGTGGCTGGCTGTCCCAACTCCTTGATTAAAGAG
CTCCATCACTTCAGGATTCTGGGAGAAGAGCAGTACAACCGGTACCAGCA
GTATGGTGCAGAGGAGTGTGTCCTGCAGATGGGGGGCGTGTTATGCCCCC
GCCCTGGCTGTGGAGCGGGGCTGCTGCCGGAGCCTGACCAGAGGAAAGTC
ACCTGCGAAGGGGGCAATGGCCTGGGCTGTGGGTTTGCCTTCTGCCGGGA
ATGTAAAGAAGCGTACCATGAAGGGGAGTGCAGTGCCGTATTTGAAGCCT
CAGGAACAACTACTCAGGCCTACAGAGTCGATGAAAGAGCCGCCGAGCAG
GCTCGTTGGGAAGCAGCCTCCAAAGAAACCATCAAGAAAACCACCAAGCC
CTGTCCCCGCTGCCATGTACCAGTGGAAAAAAATGGAGGCTGCATGCACA
TGAAGTGTCCGCAGCCCCAGTGCAGGCTCGAGTGGTGCTGGAACTGTGGC
TGCGAGTGGAACCGCGTCTGCATGGGGGACCACTGGTTCGACGTGTAG

>wild-type AAV2 ITR nucleic acid sequence (SEQ ID NO: 5)

AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGAGAGGGAGTGGCCAA

What is claimed is:

1. An isolated nucleic acid comprising an expression construct encoding a human Parkin protein (PRKN), wherein the human PRKN protein is encoded by a codon-optimized nucleic acid sequence.

2. The isolated nucleic acid of claim 1, wherein the human PRKN protein comprises the amino acid sequence set forth in SEQ ID NO: 1, or a portion thereof.

3. The isolated nucleic acid of claim 1 or 2, wherein the codon-optimized nucleic acid sequence encoding the human PRKN protein comprises the nucleic acid sequence set forth in SEQ ID NO: 2 or 3.

4. The isolated nucleic acid of any one of claims 1 to 3, wherein the expression construct further comprises a promoter operably linked to the codon-optimized nucleic acid sequence.

5. The isolated nucleic acid of claim 4, wherein the promoter is a constitutive promoter, inducible promoter, or tissue-specific promoter.

6. The isolated nucleic acid of claim 5, wherein the promoter is a chicken beta-actin (CBA) promoter, a CAG promoter, or a JeT promoter.

7. The isolated nucleic acid of any one of claims 1 to 6, wherein the expression construct is flanked by adeno-associated virus (AAV) inverted terminal repeats (ITRs).

8. The isolated nucleic acid of claim 7, wherein the AAV ITRs are of a serotype selected from the group consisting of AAV1 ITR, AAV2 ITR, AAV3 ITR, AAV4 ITR, AAV5 ITR, and AAV6 ITR.

9. The isolated nucleic acid of claim 8, wherein the AAV ITRs are AAV2 ITRs.

10. A vector comprising the isolated nucleic acid of any one of claims 1 to 9.

11. The vector of claim 10, wherein the vector is a plasmid.

12. The vector of claim 10, wherein the vector is a viral vector, optionally wherein the viral vector is a recombinant AAV (rAAV) vector or a Baculovirus vector.

13. A host cell comprising the isolated nucleic acid of any one of claims 1 to 9 or the vector of any one of claims 10-12.

14. The host cell of claim 13, wherein the host cell is a mammalian cell, yeast cell, bacterial cell, or insect cell, optionally wherein the host cell is a human cell.

15. A recombinant adeno-associated virus (rAAV) comprising:
(i) a capsid protein; and
(ii) the isolated nucleic acid of any one of claims 1 to 9, or the vector of any one of claims 10 to 12.

16. The rAAV of claim 15, wherein the capsid protein is capable of crossing the blood-brain barrier.

17. The rAAV of claim 16, wherein the capsid protein is an AAV9 capsid protein or a variant thereof.

18. The rAAV of claim 16 or 17, wherein the rAAV transduces neuronal cells and/or non-neuronal cells of the central nervous system (CNS).

19. A composition comprising the isolated nucleic acid of any one of claims 1 to 9, the vector of any one of claims 10-12, the host cell of any one of claims 13 to14, or the rAAV of any one of claims 15 to 18.

20. The composition of claim 19, wherein the composition is a pharmaceutical composition, and wherein the pharmaceutical composition further comprises a pharmaceutically acceptable carrier.

21. A method for delivering a transgene to cells of the central nervous system, the method comprising administering the rAAV of any one of claims 14 to 18 to a subject.

22. The method of claim 21, wherein the administration is direct injection into CNS tissue.

23. The method of claim 21, wherein the administration is peripheral administration, optionally wherein the peripheral administration is intravenous injection.

24. A method for treating a subject having or suspected of having Parkinson’s disease, the method comprising administering to the subject the isolated nucleic acid of any one of claims 1 to 9, the vector of any one of claims 10 to 12, the host cell of any one of claims 13 to 14, the rAAV of any one of claims 15 to 18, or the composition of any one of claims 19 to 20.

25. The method of claim 24, wherein the administration comprises direct injection to the CNS of the subject, optionally wherein the direct injection is intracerebral injection, intraparenchymal injection, intrathecal injection, intra-cisterna magna (ICM) injection or any combination thereof.

26. The method of claim 25, wherein the direct injection to the CNS of the subject comprises convection enhanced delivery (CED).

27. The method of any one of claims 24 to 26, wherein the administration comprises peripheral injection, optionally wherein the peripheral injection is intravenous injection.

28. The method of any one of claims 24 to 27, wherein the subject comprises a mutation in a PRKN gene.

29. The method of claim 28, wherein the mutation in PRKN gene is comprises a nucleotide substitution, deletions, or splice site mutation.

30. A recombinant adeno-associated virus (AAV) vector comprising a nucleic acid comprising, in 5′ to 3′ order:
(a) a 5′ AAV ITR;
(b) a CMV enhancer;
(c) a CBA promoter;
(d) a transgene encoding a PRKN protein, wherein the PRKN protein is encoded by the nucleic acid sequence in SEQ ID NO: 2 or 3;
(e) a WPRE;
(f) a Bovine Growth Hormone polyA signal tail; and
(g) a 3′ AAV ITR.

31. A recombinant adeno-associated virus (rAAV) comprising:
(i) an AAV capsid protein; and
(ii) the rAAV vector of claim 30.

32. The rAAV of claim 31, wherein the AAV capsid protein is AAV9 capsid protein.

33. A plasmid comprising the rAAV vector of claim 30.

34. A Baculovirus vector comprising the nucleic acid sequence set forth in SEQ ID NO: 2 or 3.

35. A cell comprising:
(i) a first vector encoding one or more adeno-associated virus rep protein and/or one or more adeno-associated virus cap protein; and
(ii) a second vector comprising the nucleic acid sequence set forth in SEQ ID NO: 2 or 3.

36. The cell of claim 35, wherein the first vector is a plasmid and the second vector is a plasmid.

37. The cell of claim 35, wherein the cell is a mammalian cell, optionally wherein the mammalian cell is a HEK293 cell.

38. The cell of claim 35, wherein the first vector is a Baculovirus vector and the second vector is a Baculovirus vector.

39. The cell of claim 38, wherein the cell is an insect cell, optionally wherein the insect cell is a SF9 cell.

40. A method of producing the rAAV of claim 31 or 32, the method comprising:
(i) delivering to a cell a first vector encoding one or more adeno-associated virus rep protein and/or one or more adeno-associated cap protein, and a recombinant AAV vector comprising the nucleotide sequence of SEQ ID NO: 2 or 3;
(ii) culturing the cells under conditions allowing for packaging the rAAV; and
(iii) harvesting the cultured host cell or culture medium for collection of the rAAV.

41. A method for treating a subject having or suspected of having Parkinson’s disease, the method comprising administering to the subject the rAAV of claim 31 or 32.

42. The method of claim 41, wherein the administration comprises direct injection to the CNS of the subject, optionally wherein the direct injection is intracerebral injection, intraparenchymal injection, intrathecal injection, intra-cisterna magna injection or any combination thereof.

43. The method of claim 42, wherein the direct injection to the CNS of the subject comprises convection enhanced delivery (CED).

44. The method of any one of claims 41, wherein the administration comprises peripheral injection, optionally wherein the peripheral injection is intravenous injection.

45. A method for correcting mitochondrial dysfunction in a cell, the method comprising contacting the cell with the isolated nucleic acid of any one of claims 1 to 9, the vector of any one of claims 10-12, or the rAAV of any one of claims 15 to 18.

46. The method of claim 45, wherein the contacting comprises contacting the cell with an amount of the isolated nucleic acid, vector, or rAAV in an amount sufficient to reduce oxidative stress in the cell and/or increase mitophagy in the cell.

47. The method of claim 45 or 46, wherein the cell is a mammalian cell.

48. The method of any one of claims 45 to 47, wherein the cell is a human cell.

49. The method of any one of claims 45 to 48, wherein the cell comprises one or more mutations, insertions, or deletions in a PRKN gene.

50. The method of any one of claims 45 to 49, wherein the cell is in vitro.

51. The method of any one of claims 45 to 49, wherein the cell is in a subject.

52. The method of claim 51, wherein contacting the cell in a subject is by administering to the subject the isolated nucleic acid of any one of claims 1 to 9, the vector of any one of claims 10-12, or the rAAV of any one of claims 15 to 18, via peripheral injection, optionally wherein the peripheral injection is intravenous injection.

53. The method of any one of claims 45 to 52, wherein after the contacting occurs, mitochondrial dysfunction is reduced in the cell by at least 1% relative to the mitochondrial dysfunction in the cell prior to the contacting.

54. The method of any one of claims 41 to 44, wherein the subject is a non-human mammal.

55. The method of any one of claims 41 to 44, wherein the subject is a human subject.
